(19) United States
(12) Patent Application Publication
(10) Pub. No.: US 2018/0243273 A1
(43) Pub. Date: Aug. 30, 2018

(54) Composition for Intravesical Administration for Treating Bladder Pain

(71) Applicant: Gruenenthal GmbH, Aachen (DE)

(72) Inventor: Carlos VAN HEMELRIJCK, Aachen (DE)

(21) Appl. No.: 15/903,772

(22) Filed: Feb. 23, 2018

(30) Foreign Application Priority Data
Feb. 24, 2017 (EP) 17 020 068.7

(51) Int. Cl. A61K 31/438; A61K 9/08; A61K 47/10; A61K 47/22

(52) U.S. Cl. CPC A61K 31/438; A61K 47/22; A61K 47/10; A61K 9/08

(57) ABSTRACT

The invention relates to a pharmaceutical composition comprising cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine. The pharmaceutical composition is suitable for topical administration, especially for intravesical administration in the treatment of bladder pain.

The invention relates to a pharmaceutical composition comprising cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine. The pharmaceutical composition is suitable for topical administration, especially for intravesical administration in the treatment of bladder pain.

Bladder pain syndrome (BPS), also known as interstitial cystitis, is a type of chronic pain that affects the bladder. Symptoms include feeling the need to urinate right away and often, and pain with sex. BPS is associated with depression and lower quality of life. Many patients suffering from BPS have irritable bowel syndrome and fibromyalgia, too.

The cause of BPS is unknown and there is no cure for BPS. Conventional treatments that may improve symptoms include lifestyle changes, medications, or procedures. Lifestyle changes may include stopping smoking and reducing stress. Medications may include ibuprofen, pentosan polysulfate, or amitriptyline. Procedures may include bladder distention, nerve stimulation, or surgery.

The American Urological Association released consensus-based guideline for the diagnosis and treatment of BPS including the following treatments:
1st-line treatments: patient education, self care (diet modification), stress management;
2nd-line treatments: physical therapy, oral medications (amitryptiline, cimetidine or hydroxyzine, pentosan polysulfate), bladder instillations (DMSO, heparin, or lidocaine);
3rd-line treatments: treatment of Hunner's ulcers (laser, fulguration or triamcinolone injection), hydrodistention (low pressure, short duration);
4th-line treatments: neuromodulation (sacral or pudendal nerve);
5th-line treatments: cyclosporine A, botulinum toxin (BTX-A); and
6th-line treatments: surgical intervention (urinary diversion, augmentation, cystectomy)

The AUA guidelines also listed several discontinued treatments, including: long-term oral antibiotics, intravesical bacillus Calmette Guerin, intravesical resiniferatoxin), high-pressure and long-duration hydrodistention, and systemic glucocorticoids.

Bladder instillation of medication is one form of treatment of BPS. Single medications or a mixture of medications are commonly used in bladder instillation preparations. Such preparations are typically aqueous and must be prepared under conditions ensuring sterility of the final product.

Agents used for bladder instillations to treat BPS include: DMSO, heparin, lidocaine, chondroitin sulfate, hyaluronic acid, pentosan polysulfate, oxybutynin, and botulinum toxin A. Preliminary evidence suggests these agents are efficacious in reducing symptoms of BPS. Amitriptyline has been shown to be effective in reducing symptoms such as chronic pelvic pain and nocturia in many patients with BPS. The antidepressant duloxetine was found to be ineffective as a treatment, although it is known to relieve neuropathic pain (Ch. Papandreou et al. Advances in Urology. 2009: 1-9). The calcineurin inhibitor cyclosporine A has been studied as a treatment for BPS due to its immunosuppressive properties. A prospective randomized study found cyclosporine A to be more effective at treating BPS symptoms than pentosan polysulfate, but also had more adverse effects. Oral pentosan polysulfate is believed to repair the protective glycosaminoglycan coating of the bladder, but studies have encountered mixed results when attempting to determine if the effect is statistically significant compared to placebo.

The treatment options for BPS according to the prior art are not satisfactory in every respect and there is a demand for new medicaments for treating BPS.

The pharmacologically active ingredient cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′ [1H]-pyrido[3,4-b]indol]-4-amine is an analgesic known from WO 2012/013343.

Cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine is poorly soluble in water and even in the presence of conventional solubility enhancers, concentrations in aqueous solution are low. Further, cis-(E)-4-(3-fluorophenyl)-2′,3′,4′,9′-tetrahydro-N,N-dimethyl-2′-(1-oxo-3-phenyl-2-propenyl)-spiro-[cyclohexane-1,1′[1H]-pyrido[3,4-b]indol]-4-amine is sensitive towards chemical decomposition such that aqueous solutions have poor storage stability and short shelf-life.

It is an object of the invention to provide pharmaceutical compositions that are useful for ameliorating conditions and symptoms that are associated with interstitial cystitis, especially for treating bladder pain syndrome (pain due to interstitial cystitis) and that have advantages compared to the prior art. Further, it is an object of the invention to provide pharmaceutical compositions of cis-(E)-4-(3-fluorophenyl)-2′,3′,4′,9′-tetrahydro-N,N-dimethyl-2′-(1-oxo-3-phenyl-2-propenyl)-spiro-[cyclohexane-1,1′[1H]-pyrido[3,4-b]indol]4-amine or its physiologically acceptable salts that are useful for topical administration, preferably intravesical administration, and that have advantages compared to the prior art. The pharmaceutical compositions should contain cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]4-amine in dissolved form at sufficiently high concentration, should comply with requirements for sterile formulations, and should have a sufficient storage stability and shelf-life.

These objects have been achieved by the subject-matter of the patent claims.

### BRIEF DESCRIPTION OF THE DRAWINGS

FIG. 1—Solubility and impurities of API in presence of solubility enhancing excipients in citrate buffer.

FIG. 2—Solubility and impurities of API in presence of solubility enhancing excipients in phosphate buffer.

FIG. 3—Results of the stability assay in citrate buffer and SLS or vitamin E TPGS in dependence of light protection.

FIG. 4—Purity in citrate buffer and SLS or vitamin E TPGS in dependence of light protection.

FIG. 5—Results of the stability assay in phosphate buffer and Labrasol® or vitamin E TPGS in dependence of light protection.

FIG. 6—Purity in phosphate buffer and Labrasol® or vitamin E TPGS in dependence of light protection.

FIG. 7—Impurities in citrate buffer and SLS or vitamin E TPGS as well as phosphate buffer and Labrasol® or vitamin E TPGS after autoclaving.

FIG. 8—Results of the assay and impurities in citrate buffer, 0.25 and 0.5 SLS and vitamin E TPGS, each.

FIG. 9—Results of the assay and impurities in phosphate buffer, 0.25 and 0.5% Labrasol® and vitamin E TPGS, each.

FIG. 10—Results of the assay and impurities in citrate buffer and SLS (0.1%; 0.25%; 0.5% and 1.0%) in presence and absence of ascorbic acid.

FIG. 11—pH in citrate buffer and SLS (0.1%; 0.25%; 0.5% and 1.0%) in presence and absence of ascorbic acid (+++=very cloudy; ++=cloudy; −=clear; (*)=yellow color).

FIG. 12—Results of the assay and impurities in phosphate buffer and vitamin E TPGS (0.1%; 0.25%; 0.5% and 1.0%) in presence and absence of ascorbic acid.

FIG. 13—pH in phosphate buffer and vitamin E TPGS (0.1%; 0.25%; 0.5% and 1.0%) in presence and absence of ascorbic acid (+=slightly cloudy; −=clear; (″)=visible crystals).

FIG. 14—Short term stability of API in phosphate buffer and vitamin E TPGS 0.5% and 1% in presence and absence of ascorbic acid; Assay at t=6, 14 and 21 days.

FIG. 15—Short term stability of API in phosphate buffer and vitamin E TPGS 0.5% and 1% in presence and absence of ascorbic acid; Impurities at t=6, 14 and 21 days.

FIG. 16—Short term stability of API in phosphate buffer and vitamin E TPGS 0.5% and 1% in presence and absence of ascorbic acid; pH and appearance at t=6, 14 and 21 days (−=clear; (*)=yellow color).

FIG. 17—Results of the assay, impurities and pH of API in phosphate buffer and vitamin E TPGS 0.5% in presence and absence of ascorbic acid.

FIG. 18—Flow chart for assessment of the influence of nitrogen-gassing.

It has been surprisingly found that pharmaceutical compositions can be prepared which contain cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine or its physiologically acceptable salts (in the following also referred to as “API”) at sufficiently high concentrations, and which are useful for ameliorating conditions and symptoms that are associated with interstitial cystitis, especially for treating bladder pain syndrome (pain due to interstitial cystitis). The pharmaceutical compositions according to the invention can be provided as stable sterile compositions, which are well tolerated by the patient after intravesical application.

In spite of the low solubility of the API in water, solubility enhancing excipients have been found that may be incorporated to the solution.

It has been surprisingly found that certain excipient and buffer combinations are useful to prepare aqueous pharmaceutical compositions of the API with acceptable recovery and stability properties. Further, it has been surprisingly found that the stability of the API is a function of the excipient concentration, whereas the solubility of the API is a function of the pH value and of the excipient concentration. Further, it has been surprisingly found that the API is subject to light-induced degradation and that amber glass containers have advantages compared to other primary packaging materials.

To enhance the oxidative resistance of the composition, the presences of ascorbic acid as an antioxidant and nitrogen as protective gas were assessed. However, both the presence of ascorbic acid and nitrogen lead to no evidence to increase stability. Ascorbic acid necessitates pH adjustment due to the occurrence of a pH shift and furthermore, results in negative effects on the stability at 25° C.

The stability of the pharmaceutical composition was assessed by means of autoclaving experiments, where the compositions were treated at 121° C. and 2 bar for 20 min.

Furthermore, it has been found that by employing micronized API, advantageous pharmaceutical compositions can be prepared, particularly with respect to improved dissolution rate of the API. The process for the preparation of the pharmaceutical composition may be carried out under aseptic conditions, preferably by preparing a melt of the API and excipient, by subsequently adding aqueous buffer to the melt, and by filtration through a membrane filter.

A first aspect of the invention relates to an aqueous pharmaceutical composition comprising cis-(E)-4-(3-fluorophenyl)-2′,3′,4′,9′-tetrahydro-N,N-dimethyl-2′-(1-oxo-3-phenyl-2-propenyl)-spiro-[cyclohexane-1,1′[1H]-pyrido[3,4-b]indol]4-amine or a physiologically acceptable salt thereof at a concentration of at least 5.0 μg/mL, more preferably at least 10 μg/mL, more preferably at least 20 μg/mL.

The pharmaceutical composition according to the invention contains the API cis-(E)-4-(3-fluorophenyl)-2′,3′,4′,9′-tetrahydro-N,N-dimethyl-2′-(1-oxo-3-phenyl-2-propenyl)-spiro[cyclo-hexane-1,1′[1H]-pyrido[3,4-b]indol]4-amine having the following structure

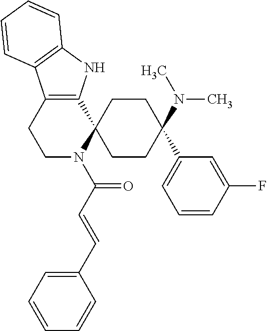 [SMILES: CN(C)[C@]1(c2cccc(F)c2)CC[C@]2(CC1)c1[nH]c3ccccc3c1CCN2C(=O)/C=C/c1ccccc1]

or a physiologically acceptable salt thereof.

Physiologically acceptable salts of the API include but are not limited to the citrate salt and the hydrochloride salt. Preferably, the API is contained in the pharmaceutical composition in the non-salt form, i.e. in form of its free base. Nonetheless, a skilled person recognizes that depending upon the pH value of the pharmaceutical composition and its constituents, acid addition salts may form in situ. In the course of the preparation of the pharmaceutical composition according to the invention, the API is preferably added in the non-salt form, i.e. in form of its free base.

Unless expressly stated otherwise, all percentages are wt.-%. Further, unless expressly stated otherwise, all weights and percentages of the API are expressed in terms of equivalents relative to the weight of the non-salt form of the API. Unless expressly stated otherwise, all properties are determined at 50% relative humidity and 23° C.

The pharmaceutical composition according to the invention is aqueous. Preferably, the pharmaceutical composition is liquid at room temperature, preferably a liquid of low viscosity. Preferably, the water content of the pharmaceutical composition is at least 90 wt.-%, more preferably at least 95 wt.-%, and most preferably at least 97 wt.-%, in each case relative to the total weight of the composition.

Besides water, the composition according to the invention may contain further solvents. Further suitable solvents include all types of physiologically acceptable hydrophilic solvents, preferably selected from the group consisting of ethanol, glycerol, propylene glycol, 1,3-butanediol and macrogol 300.

Preferably, however, water is the only solvent that is contained in the pharmaceutical composition according to the invention.

Preferably, the pharmaceutical composition according to the invention is suitable for topical administration, preferably intravesical administration, and hence satisfies the regulatory requirements for such compositions. Preferably, the pharmaceutical composition has been prepared under aseptic conditions and hence can be regarded as sterile.

The pharmaceutical composition according to the invention contains the API at a concentration of at least 5.0 μg/mL, more preferably at least 10 μg/mL, more preferably at least 20 μg/mL.

The pharmaceutical composition may contain the API in dissolved form, dispersed form (suspended and/or emulsified), or combinations thereof. For the purpose of the specification, the concentration relates to the quantity of the API that is contained in a non-solid, preferably liquid aqueous phase of the composition. Preferably, the composition consists of such a liquid aqueous phase.

Thus, in case that the pharmaceutical composition should be e.g. a saturated solution in form of an aqueous overhead solution (liquid aqueous phase) above a precipitate of API (solid phase), only the factually dissolved (or dispersed) quantity of the API that is contained in the liquid aqueous phase contributes to the concentration. In case that the pharmaceutical composition should be e.g. a suspension, wherein API is suspended in a liquid aqueous phase, the amount of the suspended API contributes to the concentration. Likewise, in case that the pharmaceutical composition should be e.g. an emulsion, wherein API is emulsified in a liquid aqueous phase, the amount of the emulsified API contributes to the concentration.

Preferably, the total quantity of the API that is contained in the pharmaceutical composition according to the invention is dissolved at 23° C.

Preferably, at 23° C. the pharmaceutical composition is clear, i.e. non-cloudy or non-opaque, upon inspection with the naked eye.

In preferred embodiments, the concentration of the API in the pharmaceutical composition is at least 30 μg/mL, or at least 40 μg/mL, or at least 50 μg/mL, or at least 60 μg/mL, or at least 70 μg/mL, or at least 80 μg/mL, or at least 90 μg/mL, or at least 100 μg/mL, or at least 110 μg/mL, or at least 120 μg/mL, or at least 130 μg/mL, or at least 140 μg/mL, or at least 150 μg/mL, or at least 160 μg/mL, or at least 170 μg/mL, or at least 180 μg/mL, or at least 190 μg/mL, or at least 200 μg/mL.

In preferred embodiments, the concentration of the API in the pharmaceutical composition is at most 300 μg/ml, or at most 290 μg/ml, or at most 280 μg/ml, or at most 270 μg/ml, or at most 260 μg/ml, or at most 250 μg/ml, or at most 240 μg/ml, or at most 230 μg/ml, or at most 220 μg/ml, or at most 210 μg/ml, or at most 200 μg/ml, or at most 190 μg/ml, or at most 180 μg/ml, or at most 170 μg/ml, or at most 160 μg/ml, or at most 150 μg/ml.

In preferred embodiments, the concentration of the API in the pharmaceutical composition is within the range of 40±30 μg/mL, or 60±30 μg/mL, or 80±50 μg/mL, or 80±30 μg/mL, or 100±50 μg/mL, or 100±30 μg/mL, or 120±100 μg/mL, or 120±50 μg/mL, or 120±30 μg/mL, or 140±100 μg/mL, or 140±50 μg/mL, or 140±30 μg/mL, or 160±100 μg/mL, or 160±50 μg/mL, or 160±30 μg/mL, or 180±100 μg/mL, or 180±50 μg/mL, or 180±30 μg/mL, or 200±100 μg/mL, or 200±50 μg/mL, or 200±30 μg/mL.

Preferably, the concentration of the API in the pharmaceutical composition is within the range of from 60 to 100%, more preferably 65 to 95%, still more preferably 70 to 90%, yet more preferably 75 to 85%, of the concentration of a saturated solution at 23° C. under the given conditions (same pH, same nature and content of remaining constituents). For example, when the concentration of a saturated solution of the API under the given conditions is 188 μg/mL, a range of from 60 to 100% of the concentration of said saturated solution means a concentration within the range of from 112.8 μg/mL (i.e. 60% of 188 μg/mL) to 188 μg/mL (i.e. 100% of 188 μg/mL).

In preferred embodiments, the pharmaceutical composition according to the invention has a pH value of at least pH 2.0, or at least pH 2.5, or at least pH 3.0, or at least pH 3.5, or at least pH 4.0, or at least pH 4.5.

In preferred embodiments, the pharmaceutical composition according to the invention has a pH value of at most pH 8.0, or at most pH 7.5, or at most pH 7.0, or at most pH 6.5, or at most pH 6.0, or at most pH 5.5.

Preferably, the pH value of the pharmaceutical composition is within the range of from pH 2.0 to pH 12, more preferably from pH 2.5 to pH 8; still more preferably from pH 3.0 to pH 7.0; yet more preferably from pH 3.5 to pH 6.5, most preferably from pH 4.0 to pH 6.0, and in particular from pH 4.5 to pH 5.5.

It has been surprisingly found that pH values within the range of from about pH 4 to about pH 6 provide a particularly beneficial compromise between solubility of the API on the one hand and its chemical stability on the other hand.

Preferably, the composition according to the invention is buffered, i.e. contains one or more buffers and buffer systems (i.e. conjugate acid-base-pairs), respectively. Preferred buffer systems are derived from the following acids: organic acids such as acetic acid, propionic acid, maleic acid, fumaric acid, lactic acid, malonic acid, malic acid, mandelic acid, citric acid, tartaric acid, succinic acid; or inorganic acids such as phosphoric acid. When the buffer systems are derived from any of the above acids, the buffer system constitutes of said acid and its conjugate base. Buffer systems derived from acetic acid, citric acid, lactic acid, succinic acid or phosphoric acid are particularly preferred, a buffer derived from phosphoric acid is especially preferred.

It has been surprisingly found that at the same pH value, a buffer derived from phosphoric acid (phosphate buffer) provides advantages compared to a buffer derived from citric acid (citrate buffer).

A skilled person is fully aware that multiprotonic acids can form more than a single buffer system. For example, phosphoric acid is a triprotonic acid so that it forms the conjugate acid-base pairs phosphoric acid—dihydrogen phosphate, dihydrogen phosphate—hydrogen phosphate and hydrogen phosphate—phosphate. In other words, any of phosphoric acid, dihydrogen phosphate and hydrogen phosphate can be the acid of a buffer system with the conjugate base. For the purpose of the specification, the expression “buffer and buffer system, respectively” preferably refers to the quantity of both, the acid and its conjugate base. Further, a skilled person is fully aware that a buffer system, e.g. the conjugate system phosphoric acid/potassium dihydrogen phosphate can be established either by adding phosphoric acid and an appropriate amount of potassium hydroxide, or potassium phosphate and an appropriate amount of phosphoric acid, or phosphoric acid and potassium dihydrogen phosphate as such.

Preferably, the concentration of the buffer and buffer system, respectively, preferably derived from phosphoric acid, is adjusted to provide a sufficient buffer capacity.

In a preferred embodiment, the content of the buffer and buffer system, respectively, preferably derived from phosphoric acid, is within the range of from 0.0001 to 5.0 wt.-%, more preferably 0.0002 to 2.5 wt.-%, still more preferably 0.0005 to 1.0 wt.-%, yet more preferably 0.001 to 0.5 wt.-%, most preferably 0.005 to 0.25 wt.-% and in particular 0.01 to 0.1 wt.-%, based on the total weight of the composition.

The pharmaceutical composition according to the invention preferably comprises an excipient selected from antioxidants, surfactants and surfactants having antioxidative properties (antioxidants having amphiphilic properties). Thus, the excipient may serve more than one purpose. In one embodiment, the pharmaceutical composition comprises an antioxidant and/or a surfactant, which differ from one another. In another embodiment, the pharmaceutical composition comprises one excipient which is a surfactant having antioxidative properties (i.e. can alternatively be regarded as an antioxidant having amphiphilic properties).

For the purpose of the specification, the term “surfactant” refers to any compound that has amphiphilic properties, as it contains at least one hydrophobic group and at least one hydrophilic group. Preferably, a surfactant contains at least one terminal hydrophobic group (tail) and at least one terminal hydrophilic group (head). The hydrophobic group is preferably selected from the group consisting of hydrocarbon, alkyl ether, fluorocarbon and siloxane groups.

In a preferred embodiment, the excipient contains at least one aliphatic group comprising at least 3 carbon atoms, more preferably at least 4 carbon atoms, still more preferably at least 6 carbon atoms, yet more preferably 6 to 30 carbon atoms, and most preferably 8 to 24 carbon atoms. The aliphatic group may be a saturated or unsaturated, branched or unbranched (linear), terminal or internal aliphatic group.

Preferably, the excipient comprises a polyethylene glycol residue.

Preferably, the excipient contains at least one group derivable from a saturated or unsaturated fatty acid or from a saturated or unsaturated fatty alcohol, which group is preferably an ether, carboxylic acid ester or sulfuric acid ester group. Preferably, the saturated or unsaturated fatty acid or fatty alcohol contains at least 6 carbon atoms, yet more preferably 6 to 30 carbon atoms, and most preferably 8 to 24 carbon atoms.

In a preferred embodiment, the excipient contains at least one group derivable from a saturated or unsaturated fatty acid, preferably C₆ to C₃₀ fatty acid, more preferably C₈ to C₂₄ fatty acid, and most preferably C₁₂ to C₂₂ fatty acid. Examples for suitable fatty acids are lauric acid, myristic acid, palmitic acid, stearic acid, arachidic acid, behenic acid, lignoceric acid, 12-hydroxy stearic acid, oleic acid and ricinoleic acid.

In another preferred embodiment, the excipient contains at least one group derivable from a saturated or unsaturated fatty alcohol, preferably C₆ to C₃₀ fatty alcohol, more preferably C₈ to C₂₄ fatty alcohol, and most preferably C₁₂ to C₂₂ fatty alcohol. Examples for suitable fatty alcohols are cetyl alcohol, stearyl alcohol, 2-octyldodecane-1-ol and 2-hexyldecane-1-ol.

Preferably, the excipient has a molecular weight of at most 20,000 g/mol, more preferably at most 15,000 g/mol, still more preferably at most 10,000 g/mol, yet more preferably at most 5,000 g/mol, even more preferably at most 4,000 g/mol, most preferably at most 3,000 g/mol, and in particular within the range of from 100 g/mol to 2,500 g/mol, preferably 1000 to 2000 g/mol.

In a preferred embodiment, the pharmaceutical composition contains a single excipient. In another preferred embodiment, the pharmaceutical composition contains a mixture of two or more excipients.

Preferably, the pharmaceutical composition contains an excipient having a hydrophilic-lipophilic balance (HLB) of at least 8 or at least 9. More preferably, the hydrophilic-lipophilic balance (HLB) is at least 10 or at least 11 or at least 12; and/or at most 18 or at most 17 or at most 16. Most preferably, the hydrophilic-lipophilic balance (HLB) ranges within 9 to 18; preferably 10 to 17, more preferably 11 to 16, and still more preferably 12 to 15.

In a preferred embodiments, the HLB value of the excipient is within the range of 10±3, or 10±2, or 10±1, or 11±3, or 11±2, or 11±1, or 12±3, or 12±2, or 12±1, or 13±3, or 13±2, or 13±1, or 14±3, or 14±2, or 14±1 or 15±3, or 15±2, or 15±1, or 16±3, or 16±2, or 16±1, or 17±3, or 17±2, or 17±1.

The excipient can be ionic, amphoteric or non-ionic.

In a preferred embodiment, the pharmaceutical composition contains an ionic excipient, in particular an anionic excipient.

Suitable anionic excipient include but are not limited to sulfuric acid esters such as sodium lauryl sulfate (sodium dodecyl sulfate, e.g. Texapon® K12), sodium cetyl sulfate (e.g. Lanette E®), sodium cetylstearyl sulfate, sodium stearyl sulfate, sodium dioctylsulfosuccinate (docusate sodium); and the corresponding potassium or calcium salts thereof.

Preferably, the anionic excipient has the general formula (I)

CₙH₂ₙ₊₁O—SO₃⁻M⁺ (I),
wherein n is an integer of from 8 to 30, preferably 10 to 24, more preferably 12 to 18; and M is selected from Li⁺, Na⁺, K⁺, NH₄⁺ ½ Mg²⁺ and ½ Ca²⁺.

Further suitable anionic excipient include salts of cholic acid including sodium glycocholate (e.g. Konakion® MM, Cernevit®), sodium taurocholate and the corresponding potassium or ammonium salts.

In another preferred embodiment, the pharmaceutical composition contains a non-ionic excipient. Suitable non-ionic excipient include but are not limited to
fatty alcohols that may be linear or branched, such as cetylalcohol, stearylalcohol, cetylstearyl alcohol, 2-octyldodecane-1-ol and 2-hexyldecane-1-ol;
sterols, such as cholesterol;
partial fatty acid esters of sorbitan such as sorbitanmonolaurate, sorbitanmonopalmitate, sorbitanmonostearate, sorbitantristearate, sorbitanmonooleate, sorbitansesquioleate and sorbitantrioleate;
partial fatty acid esters of polyoxyethylene sorbitan (polyoxyethylene-sorbitan-fatty acid esters), preferably a fatty acid monoester of polyoxyethylene sorbitan, a fatty acid diester of polyoxyethylene sorbitan, or a fatty acid triester of polyoxyethylene sorbitan; e.g. mono- and tri-lauryl, palmityl, stearyl and oleyl esters, such as the type known under the name “polysorbat” and commercially available under the trade name “Tween” including Tween® 20 [polyoxyethylene(20)-sorbitan monolaurate], Tween® 21 [polyoxyethylene(4)sorbitan monolaurate], Tween® 40 [polyoxyethylene(20)sorbitan monopalmitate], Tween® 60 [polyoxyethylene(20)sorbitan monostearate], Tween® 65 [polyoxyethylene(20)sorbitan tristearate], Tween® 80 [polyoxyethylene(20)sorbitan monooleate], Tween 81 [polyoxyethylene(5)sorbitan monooleate], and Tween® 85 [polyoxyethylene(20)sorbitan trioleate]; preferably a fatty acid monoester of polyoxyethylenesorbitan according to general formula (II)

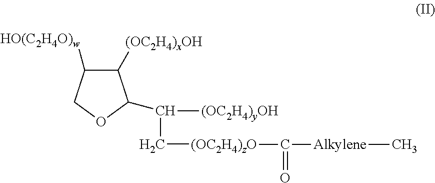

wherein (w+x+y+z) is within the range of from 15 to 100, preferably 16 to 80, more preferably 17 to 60, still more preferably 18 to 40 and most preferably 19 to 21;
and alkylene is an optionally unsaturated alkylene group comprising 6 to 30 carbon atoms, more preferably 8 to 24 carbon atoms and most preferably 10 to 16 carbon atoms;
polyoxyethyleneglycerole fatty acid esters such as mixtures of mono-, di- and triesters of glycerol and di- and monoesters of macrogols having molecular weights within the range of from 200 to 4000 g/mol, e.g., macrogolglycerolcaprylocaprate, macrogolglycerollaurate, macrogolglycerolococoate, macrogolglycerollinoleate, macrogol-20-glycerolmonostearate, macrogol-6-glycerol-caprylocaprate, macrogolglycerololeate; macrogolglycerolstearate, macrogolglycerolhydroxy-stearate (e.g. Cremophor® RH 40), and macrogolglycerolrizinoleate (e.g. Cremophor® EL);
polyoxyethylene fatty acid esters, the fatty acid preferably having from about 8 to about 18 carbon atoms, e.g. macrogololeate, macrogolstearate, macrogol-15-hydroxystearate, polyoxyethylene esters of 12-hydroxystearic acid, such as the type known and commercially available under the trade name “Solutol HS 15”; preferably according to general formula (III)

CH₃CH₂—(OCH₂CH₃)ₙ—O—CO—(CH₂)ₘCH₃ (III)

wherein n is an integer of from 6 to 500, preferably 7 to 250, more preferably 8 to 100, still more preferably 9 to 75, yet more preferably 10 to 50, even more preferably 11 to 30, most preferably 12 to 25, and in particular 13 to 20; and
wherein m is an integer of from 6 to 28; more preferably 6 to 26, still more preferably 8 to 24, yet more preferably 10 to 22, even more preferably 12 to 20, most preferably 14 to 18 and in particular 16;
polyoxyethylene fatty alcohol ethers, e.g. macrogolcetylstearylether, macrogollarylether, macrogololeylether, macrogolstearylether;
polyoxypropylene-polyoxyethylene block copolymers (poloxamers);
fatty acid esters of sucrose; e.g. sucrose distearate, sucrose dioleate, sucrose dipalmitate, sucrose monostearate, sucrose monooleate, sucrose monopalmitate, sucrose monomyristate and sucrose monolaurate;
fatty acid esters of polyglycerol, e.g. polyglycerololeate;
polyoxyethylene esters of alpha-tocopheryl succinate, e.g. D-alpha-tocopheryl-PEG-1000-succinate (TPGS);
polyglycolyzed glycerides, such as the types known and commercially available under the trade names “Gelucire 44/14”, “Gelucire 50/13 and “Labrasol”;
reaction products of a natural or hydrogenated castor oil and ethylene oxide such as the various liquid surfactants known and commercially available under the trade name “Cremophor”; and
partial fatty acid esters of multifunctional alcohols, such as glycerol fatty acid esters, e.g. mono- and tri-lauryl, palmityl, stearyl and oleyl esters, for example glycerol monostearate, glycerol monooleate, e.g. glyceryl monooleate 40, known and commercially available under the trade name “Peceol”; glycerole dibehenate, glycerole distearate, glycerole monolinoleate; ethyleneglycol monostearate, ethyleneglycol monopalmitostearate, pentaerythritol monostearate.

Especially preferred excipients of this class that are contained in the pharmaceutical composition according to the invention are non-ionic excipients having a hydrophilic-lipophilic balance (HLB) of at least 8, in particular non-ionic excipient having an HLB value of at least 9, more in particular non-ionic excipients having an HLB value within 12 and 15.

In a preferred embodiment, the content of the excipient is at least 0.001 wt.-% or at least 0.005 wt.-%, more preferably at least 0.01 wt.-% or at least 0.05 wt.-%, still more preferably at least 0.1 wt.-%, at least 0.2 wt.-%, or at least 0.3 wt.-%, yet more preferably at least 0.4 wt.-%, at least 0.5 wt.-%, or at least 0.6 wt.-%, and in particular at least 0.7 wt.-%, at least 0.8 wt.-%, at least 0.9 wt.-%, or at least 1.0 wt.-%, based on the total weight of the pharmaceutical composition.

In a preferred embodiment, the excipient is an antioxidant. Preferred antioxidants include but are not limited to ascorbic acid, butylated hydroxyanisole (BHA), butylated hydroxytoluene (BHT), salts of ascorbic acid, monothioglycerol, phosphorous acid, vitamin C, vitamin E and the derivatives thereof, coniferyl benzoate, nordihydroguajaretic acid, gallus acid esters, sodium bisulfate, particularly preferably vitamin E and the derivatives thereof.

In a preferred embodiment, the excipient is a vitamin E derivative, i.e. comprises a vitamin E residue, that is preferably linked to another residue not belonging to natural vitamin E. Preferably, said another residue is a polyethylene glycol residue which may be covalently linked to the vitamin E residue through succinate. Vitamin E derivatives (succinate diesters) of this type are also known as vitamin E polyethylene glycol succinate, which is a particularly preferred excipient according to the invention.

Vitamin E polyethylene glycol succinate is an example of an excipient according to the invention which is a surfactant having antioxidative properties (i.e. can alternatively be regarded as an antioxidant having amphiphilic properties).

The concentration of the excipient typically depends upon the desired concentration of the API in the pharmaceutical composition.

In preferred embodiments, the concentration of the excipient, preferably vitamin E polyethylene glycol succinate, is at least 0.01 wt.-%, or at least 0.05 wt.-%, or at least 0.1 wt.-%, or at least 0.2 wt.-%, or at least 0.3 wt.-%, or at least 0.4 wt.-%, or at least 0.5 wt.-%, or at least 0.6 wt.-%, or at least 0.7 wt.-%, or at least 0.8 wt.-%, or at least 0.9 wt.-%, or at least 1.0 wt.-%, or at least 1.1 wt.-%, or at least 1.2 wt.-%, or at least 1.3 wt.-%, or at least 1.4 wt.-%, or at least 1.5 wt.-%; in each case relative to the total weight of the composition.

In preferred embodiments, the concentration of the excipient, preferably vitamin E polyethylene glycol succinate, is at most 5.0 wt.-%, or at most 4.5 wt.-%, or at most 4.0 wt.-%, or at most 3.9 wt.-%, or at most 3.8 wt.-%, or at most 3.7 wt.-%, or at most 3.6 wt.-%, or at most 3.5 wt.-%, or at most 3.4 wt.-%, or at most 3.3 wt.-%, or at most 3.2 wt.-%, or at most 3.1 wt.-%, or at most 3.0 wt.-%, or at most 2.9 wt.-%, or at most 2.8 wt.-%, or at most 2.7 wt.-%, or at most 2.6 wt.-%, or at most 2.5 wt.-%.

Preferably, the concentration of the excipient, preferably vitamin E polyethylene glycol succinate, is within the range of from 0.1 to 5.0 wt.-%; preferably from 0.5 to 4.0 wt.-%, more preferably from 1.0 to 3.0 wt.-%; in each case relative to the total weight of the composition.

The pharmaceutical composition according to the invention may contain additional pharmaceutical auxiliary substances that are conventionally used in the preparation of aqueous pharmaceutical compositions and that are known to the skilled person, such as isotonizing agents, preservatives, viscosity enhancers, chelating agents, and the like.

Preferably, the composition does not contain any preservative. For the purpose of the specification, a “preservative” preferably refers to any substance that is usually added to pharmaceutical compositions in order to preserve them against microbial degradation or microbial growth. In this regard, microbial growth typically plays an essential role, i.e. the preservative serves the main purpose of avoiding microbial contamination. As a side aspect, it may also be desirable to avoid any effect of the microbes on the active ingredients and excipients, respectively, i.e. to avoid microbial degradation.

Representative examples of preservatives include benzalkonium chloride, benzethonium chloride, benzoic acid, sodium benzoate, benzyl alcohol, bronopol, cetrimide, cetylpyridinium chloride, chlorhexidine, chlorbutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, sodium propionate, thimerosal, methyl paraben, ethyl paraben, propyl paraben, butyl paraben, isobutyl paraben, benzyl paraben, sorbic acid, and potassium sorbate.

Preferably, the pharmaceutical composition according to the invention essentially consists of
water;
API;
buffer, preferably derived from phosphoric acid;
excipient, preferably vitamin E polyethylene glycol succinate; and
optionally, gases that may be dissolved in the liquid.

The pharmaceutical composition according to the invention preferably has a storage stability of at least 6 months in accordance with the ICH Guidelines, preferably the version valid in 2017.

A generally accepted accelerated test for the determination of a drug's stability according to ICH and FDA guidelines relates to the storage of a pharmaceutical composition containing the drug (e.g., in its container and packaging). According to the ICH guidelines, a so-called accelerated storage testing should be conducted for pharmaceutical compositions at 40±2° C. at 75% RH±5% for a minimum time period of 6 months. Additionally, a so-called long-term storage testing should be conducted for pharmaceutical compositions at 25±2° C. at not less than 60% RH±5% for a minimum time period of 12 months. In case that all criteria have been met for the accelerated storage testing and long-term storage testing conditions during the 6-months period, the long-time storage testing may be shortened to 6 months and the corresponding data doubled to obtain estimated data for the 12-month period.

During the storage, samples of the pharmaceutical composition are withdrawn at specified time intervals and analyzed in terms of their drug content, presence of impurities, and if applicable other parameters. According to the ICH guidelines, in all samples the purity of the drug should be ≥98%, the drug content should be 95-105% (FDA guideline: 90-110%).

In a preferred embodiment, after storage of the pharmaceutical composition for 6 months under long-term storage conditions (25° C. and 60% relative humidity) in a sealed glass container, the degradation of the API does not exceed 2.0%, more preferably 1.5%, still more preferably 1.0%, and most preferably 0.5%.

In another preferred embodiment, after storage of the pharmaceutical composition for 6 months under accelerated storage conditions (40° C. and 75% relative humidity) in a sealed glass container, the degradation of the API does not exceed 4%, more preferably 3%, still more preferably 2%, yet more preferably 1%, and most preferably 0.5%.

Another aspect of the invention relates to the aqueous pharmaceutical composition according to the invention as described above for use in the amelioration of conditions and symptoms that are associated with interstitial cystitis, especially for use in the treatment of bladder pain syndrome. In this regard, the invention also pertains to the use of the API for the manufacture of the aqueous pharmaceutical composition according to the invention as described above for use in the amelioration of conditions and symptoms that are associated with interstitial cystitis, especially for use in the treatment of bladder pain syndrome. Further, the invention also pertains to a method for ameliorating conditions and symptoms that are associated with interstitial cystitis, especially for treating bladder pain syndrome, comprising administering to a subject in need thereof the aqueous pharmaceutical composition according to the invention as described above.

Another aspect of the invention relates to cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine or a physiologically acceptable salt thereof, or to a pharmaceutical formulation comprising cis-(E)-4-(3-fluorophenyl)-2′,3′,4′,9′-tetrahydro-N,N-dimethyl-2′-(1-oxo-3-phenyl-2-propenyl)-spiro-[cyclohexane-1,1′[1H]-pyrido[3,4-b]indol]4-amine or a physiologically acceptable salt thereof, in either case for use in the amelioration of conditions and symptoms that are associated with interstitial cystitis, especially for use in the treatment of bladder pain syndrome.

Preferably, the pharmaceutical composition according to the invention is administered topically; preferably intravesically.

Preferably, the pharmaceutical composition according to the invention is administered once daily or less frequently, e.g. twice weekly or once weekly.

Another aspect of the invention relates to a container comprising the aqueous pharmaceutical composition according to the invention as described above.

Preferably, the container is a clear class container or an amber glass container, which in either case may be covered with aluminum foil.

Another aspect of the invention relates to a process for the preparation of the aqueous pharmaceutical composition according to the invention as described above comprising the steps of
(a) preparing a preblend by mixing the API with excipient at elevated temperature; and
(b) mixing the preblend obtained in step (a) with an aqueous composition, optionally containing a buffer, thereby providing the pharmaceutical composition.

Preferably, step (a) is performed at a temperature above the melting temperature of the excipient such that the preblend is a melt. Preferably, the temperature is within the range of from 50° C. to 80° C., more preferably within the range of from 55° C. to 75° C., still more preferably within the range of from 60° C. to 70° C.

Preferably, in step (a) the API is employed in micronized form. It has been surprisingly found that preparation of the aqueous pharmaceutical composition according to the invention at an industrial scale satisfactory results within satisfactory time frames can be achieved when employing the API in micronized form and preparing a preblend, preferably a melt, by mixing the API with excipient at elevated temperature, and by subsequently adding an aqueous buffer to said preblend.

Preferably, the API has a particle size distribution that is characterized by
a d10 value of at most 20 μm, preferably at most 15 μm, more preferably at most 10 μm, still more preferably at most 5.0 μm; and/or
a d50 value of at most 50 μm, preferably at most 30 μm, more preferably at most 10 μm, still more preferably at most 5.0 μm; and/or
a d90 value of at most 100 μm, preferably at most 50 μm, more preferably at most 25 μm, still more preferably at most 10 μm.

Preferably, the API has a particle size distribution that is characterized by
a d10 value within the range of from 0.15 μm to 1.05 μm, preferably within the range of from 0.30 μm to 0.90 μm, more preferably within the range of from 0.45 μm to μm 0.75; and/or
a d50 value within the range of from 0.30 μm to 2.10 μm, preferably within the range of from 0.60 μm to 1.80 μm, more preferably within the range of from 0.90 μm to 1.50 μm; and/or
a d90 value within the range of from 0.50 μm to 4.00 μm, preferably within the range of from 1.00 μm to 3.50 μm, more preferably within the range of from 1.50 μm to μm 3.00.

Suitable methods for determining the particle size distribution are known to a skilled person. Preferably the particle size distribution is determined by laser diffraction, preferably by means of a Malvern particle size analyzer, e.g. Malvern Mastersizer 3000, which is preferably operated in dry mode.

Preferably, the process according to the invention comprises the additional steps of
(c) packaging the pharmaceutical composition obtained in step (b) in a container; and
(d) optionally, autoclaving the container containing the pharmaceutical composition.

Preferably, all steps of the process according to the invention are performed under aseptic conditions.

The following examples further illustrate the invention but are not to be construed as limiting its scope:

### EXAMPLE 1: —SOLUBILIZING EFFECT OF VARIOUS EXCIPIENTS IN TWO DIFFERENT BUFFERS AT pH 4.5

The solubility of the API was assessed in different buffers together with different excipients. Batches containing different amounts of non-micronized API (i.e. 1 mg, 4 mg, 10 mg or 15 mg) in 100 g buffer were manufactured in order to have saturated solutions. Two different buffers were chosen (citrate buffer and phosphate buffer). With regard to sufficient solubility and tolerability of the final composition, a pH value of 4.5 was adjusted. In order to improve the poor solubility of the API, different solubility enhancing excipients were incorporated in the buffer systems in a concentration range of 0.1-2 wt.-%:

propylene glycol
PEG 400
lauroyl macrogol-32 glycerides (Gelucire ^{®} 44/14)
PEG-8-caprylic/capric glycerides (Labrasol ^{®})
PEG-40 hydrogenated castor oil (Cremophor ^{®} RH 40)
POE-esters of 12-hydroxy stearic acid (Solutol ^{®} HS 15)
d-alpha-tocopheryl PEG-1000 succinate (vitamin E TPGS)
polysorbate 80 (Tween ^{®} 80)
sodium lauryl sulfate (SLS)

First, buffers were prepared according to Ph. Eur. After pH adjustment, the corresponding excipient was dissolved in the buffer. 1 mg of the API was added to the excipient containing buffer (i.e. 100 g) and stirred. If the API was dissolved completely, another 9 mg were added to a final amount of 10 mg API in 100 mg buffer/excipient mixture. The resulting compositions were stirred overnight prior to filtration through a 0.45 μm filter and analysis and contained 1 mg or 10 mg API, respectively. The following compositions were prepared and the following solubility values were achieved:

| | | [mg API | Solubility | Sum of |
|---|---|---|---|---|
| buffer pH | excipient | in 100 | of API | impurities |
| 4.5 | [1 wt.-%] | g buffer] | [μg/mL] | [% (a/a)] |
| citrate | Cremophor ® RH 40 | 1 mg | 7.18 | 0.92 |
| | SLS | | 79.52 | 5.52 |
| | Solutol ® HS 15 | | 7.95 | 0.53 |
| | Tween ® 80 | | 7.93 | 1.1 |
| | SLS | 10 mg | 8.19 | 3.47 |
| | Gelucire ® 44/14 | | 34.88 | 25.91 |
| | Labrasol ® | | 12.08 | 19.81 |
| | Propylene glycol | | 0 | 0 |
| | PEG 400 | | 0 | 0 |
| | vitamin E TPGS | | 76.39 | 5.71 |
| phosphate | Cremophor ® RH 40 | 1 mg | 7.67 | 0.2 |
| | Tween ® 80 | | 8.91 | 0.52 |
| | Solutol ® HS 15 | | 6.71 | 0.2 |
| | SLS | | 1.68 | 22.55 |
| | Gelucire ® 44/14 | | 53.54 | 17.48 |
| | Labrasol ® | | 45.8 | 5.91 |
| | Propylene glycol | | 0.04 | 38.22 |
| | PEG 400 | | 0.83 | 11.7 |
| | vitamin E TPGS | 10 mg | 255.69 | 1.87 |

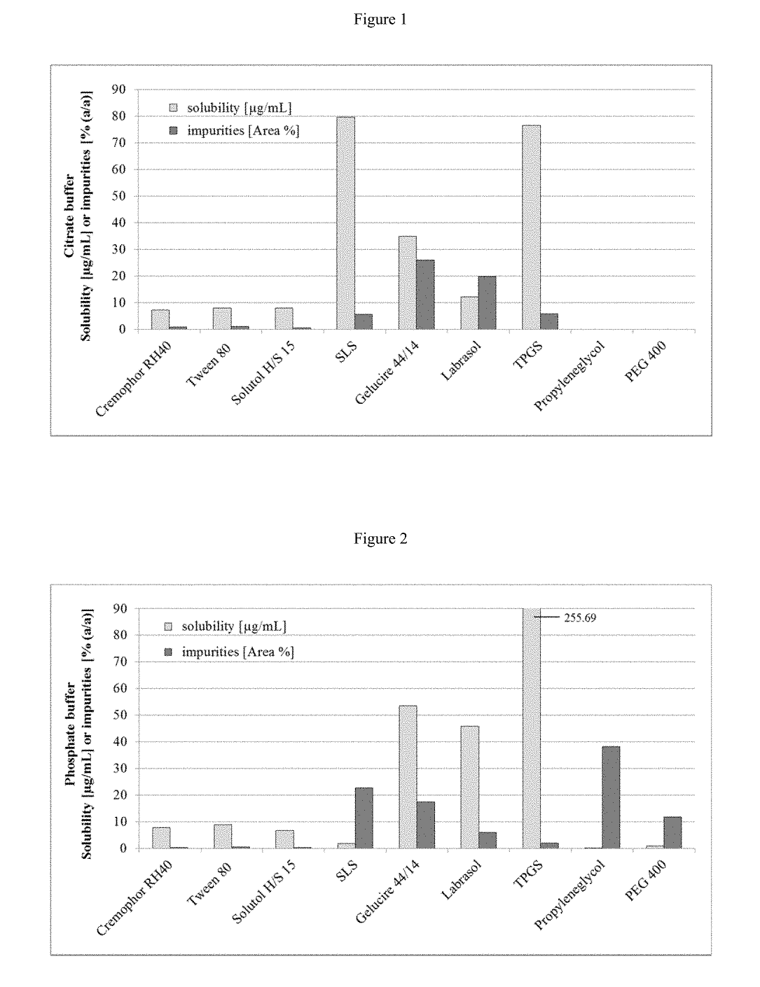

FIG. 1 shows solubility and impurities of API in presence of solubility enhancing excipients in citrate buffer. FIG. 2 shows solubility and impurities of API in presence of solubility enhancing excipients in phosphate buffer.

It becomes clear from the above data that the API showed good solubility in presence of SLS (in citrate buffer), Labrasol®, vitamin E TPGS and Gelucire® 44/14, although its impurities increased in presence of Gelucire® 44/14. The API showed good solubility in citrate buffer in presence of SLS, Gelucire® 44/14, Labrasol® and vitamin E TPGS. In phosphate buffer, good solubility was observed in Gelucire® 44/14, Labrasol® and vitamin E TPGS. Impurities of the API seemed to increase with higher solubility.

Due to the efficient solubilizing effect and lower levels of impurities, vitamin E TPGS, SLS (citrate buffer only) and Labrasol® (phosphate buffer only) were selected as surfactants for further experiments. Vitamin E TPGS has a HLB value of about 13, SLS has a HLB value of about 40, and Labrasol® has a HLB value of about 14.

### EXAMPLE 2—PHOTOSTABILITY

As preliminary studies had shown that photosensitivity is a probable reason for API degradation and since the API was stirred for several hours to obtain sufficient solubilization, the stability of API during this step was further investigated. The stirring process was investigated in dependence of three different grades of light protection: Stirring for 24 h in
clear glass containers,
amber glass containers and
clear glass containers covered with aluminum foil.

Citrate buffer containing SLS or vitamin E TPGS and phosphate buffer containing Labrasol® or vitamin E TPGS were chosen as vehicles. The compositions contained 10 mg API in 100 g buffer and were stirred for 48 h prior to filtration and analysis (additional sampling after 24 h).

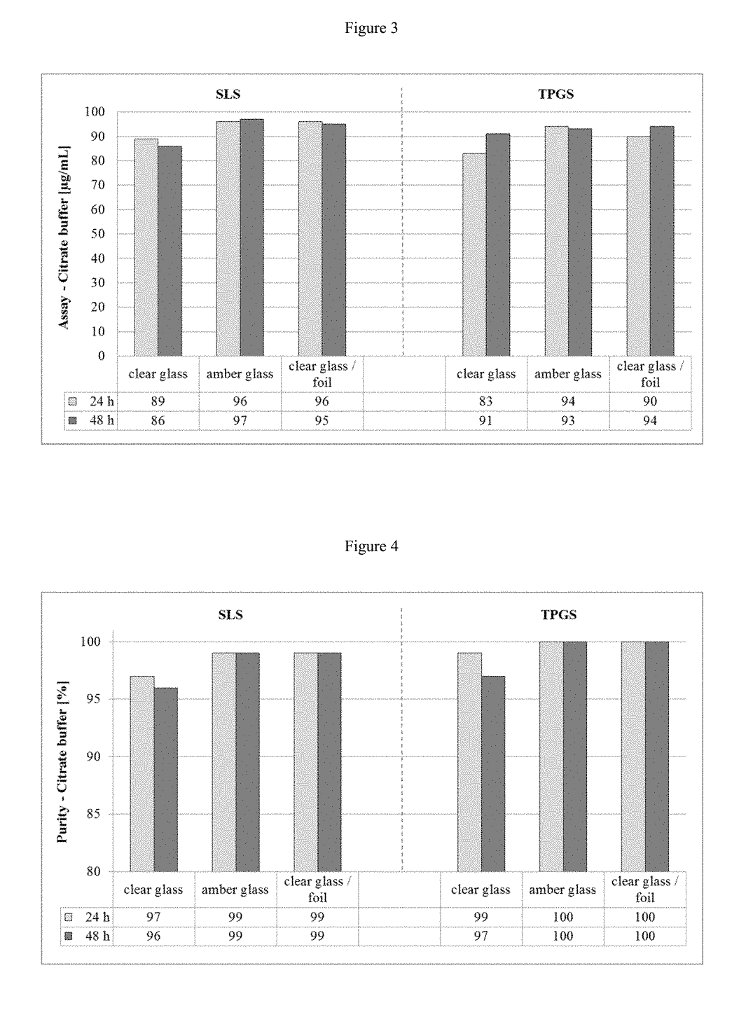

FIG. 3 shows the results of the stability assay in citrate buffer and SLS or vitamin E TPGS in dependence of light protection. FIG. 1 shows the purity in citrate buffer and SLS or vitamin E TPGS in dependence of light protection.

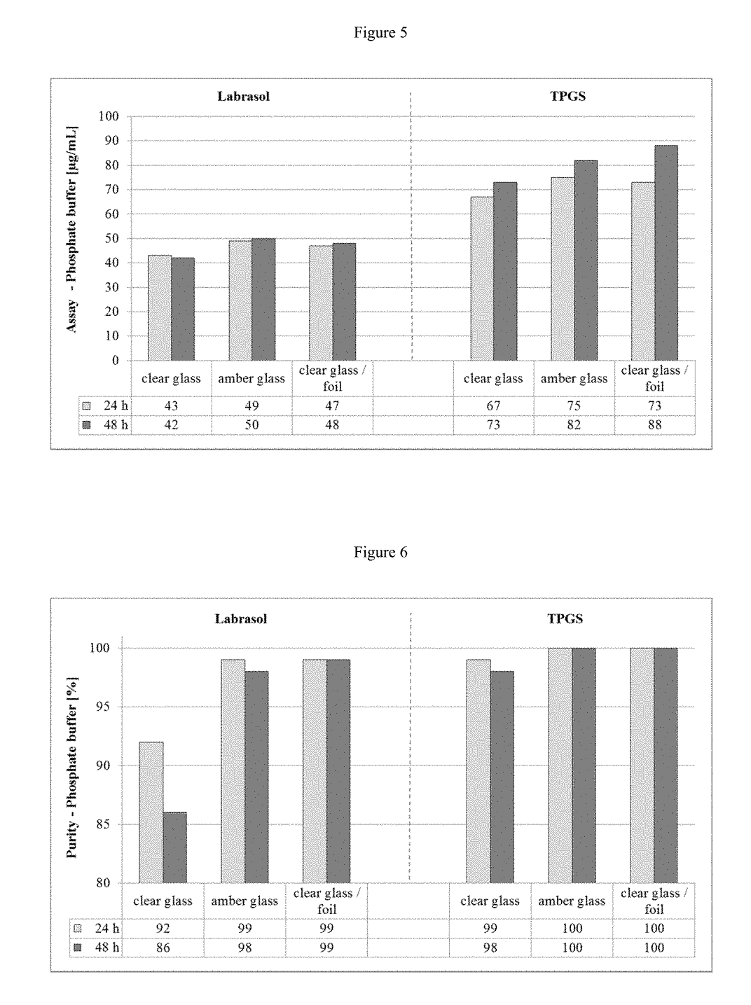

FIG. 5 shows the results of the stability assay in phosphate buffer and Labrasol® or vitamin E TPGS in dependence of light protection. FIG. 6 shows the purity in phosphate buffer and Labrasol® or vitamin E TPGS in dependence of light protection.

It becomes clear from the data shown in FIGS. 3 to 6 that in all cases light protected samples (amber glass and covered clear glass) resulted in superior assay and purity profiles. No considerable difference was noticed between amber glass and covered clear glass, indicating no general incompatibility of the API with amber glass. The usage of citrate buffer resulted in sufficient assay and purity results in combination with SLS and vitamin E TPGS, when protected from light (FIG. 3 and FIG. 4). However, the API showed significant decrease in assay when stirred in phosphate buffer and Labrasol®—even under light protection (FIG. 5). In contrast, phosphate buffer in combination with vitamin E TPGS led to sufficient assay and purity results with no considerable degradation without light protection (FIG. 5 and FIG. 6).

### EXAMPLE 3: —STABILITY AFTER AUTOCLAVING

Autoclaving experiments were performed with the aim to evaluate the stability of API in defined buffer systems and excipients. Clear glass covered by aluminium foil was chosen as primary packaging as it provided sufficient light protection for the material and was expected to provide a smaller risk of interaction than amber glass. The batches were autoclaved at 121° C. and 2 bar for 20 min, following analysis of impurity profiles.

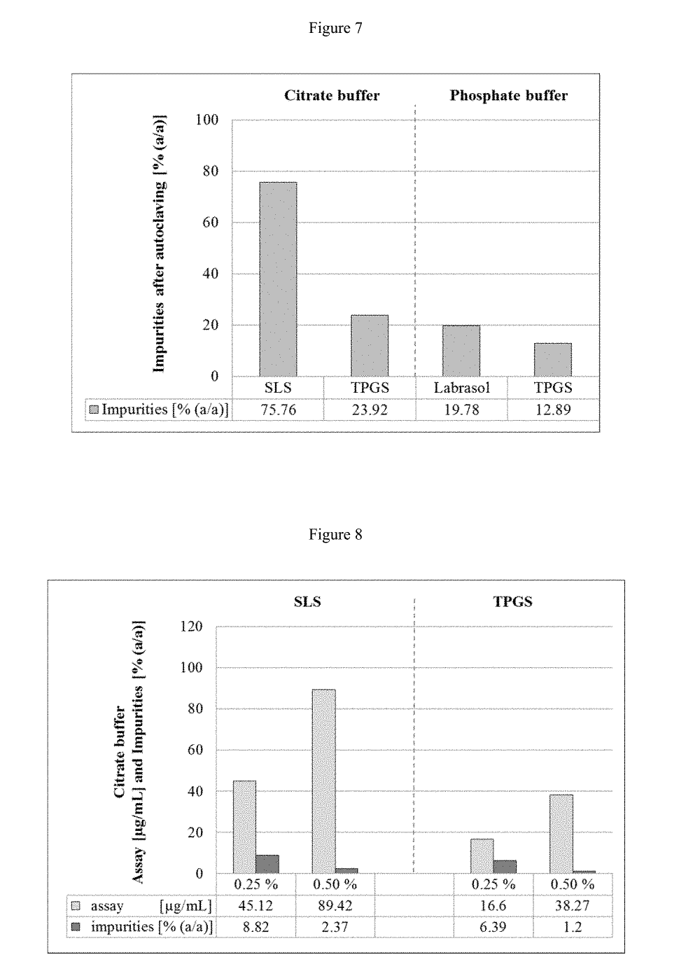

FIG. 7 shows the impurities in citrate buffer and SLS or vitamin E TPGS as well as phosphate buffer and Labrasol® or vitamin E TPGS after autoclaving.

It becomes clear from the data shown in FIG. 7 that autoclaving led to high degradation of API (sum of all impurities ranged between 13% (a/a) and 76% (a/a)), where highest degradation was observed in citrate buffer and SLS and where the API showed the lowest degradation in phosphate buffer and vitamin E TPGS.

### EXAMPLE 4—SOLUBILITY AND STABILITY OF API IN DEPENDENCE OF SURFACTANT CONCENTRATION

In order to prevent too high concentrations of surfactants, which can cause irritant or toxic effects after local administration in the bladder, lower concentrations were evaluated. New batches of API in citrate and phosphate buffer were manufactured. SLS and vitamin E TPGS were chosen for citrate buffer and Labrasol® and vitamin E TPGS for phosphate buffer in a concentration of 0.5% and 0.25%, each. The compositions were stirred overnight prior to filtration and analysis.

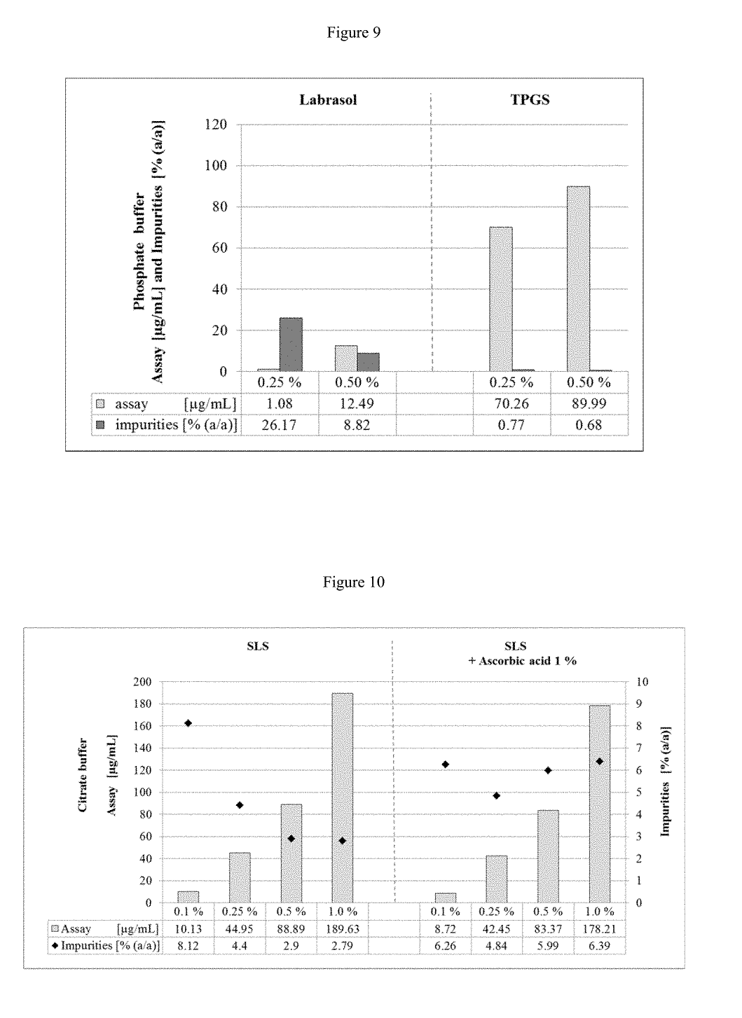

FIG. 8 shows the results of the assay and impurities in citrate buffer, 0.25 and 0.5% SLS and vitamin E TPGS, each. FIG. 9 shows the results of the assay and impurities in phosphate buffer, 0.25 and 0.5% Labrasol® and vitamin E TPGS, each.

It becomes clear from the data shown in FIGS. 8 and 9 that a considerable, surfactant concentration-dependent increase in the assay of API was observed for both buffers and all tested surfactants. In contrast, the impurities were lower at higher surfactant concentrations. The combination of phosphate buffer and 0.50% vitamin E TPGS resulted in the highest assay and lowest impurities values.

### EXAMPLE 5—SOLUBILITY AND STABILITY OF THE API IN DEPENDENCE OF SURFACTANT CONCENTRATION AND INFLUENCE OF ASCORBIC ACID AS ANTIOXIDANT

For further evaluation of the minimum concentration of surfactant required for sufficient dissolution of the API, several batches were manufactured. Citrate buffer and SLS and phosphate buffer and vitamin E TPGS were chosen with regard to the solubility and stability profiles of API. Surfactant concentrations were varied in four different steps (0.1%; 0.25%; 0.5% and 1.0%). Additionally, the influence of ascorbic acid 1% as antioxidant was assessed, since API impurities were related to oxidative degradation. The manufactured batches were stirred for six days under light protection prior to analysis.

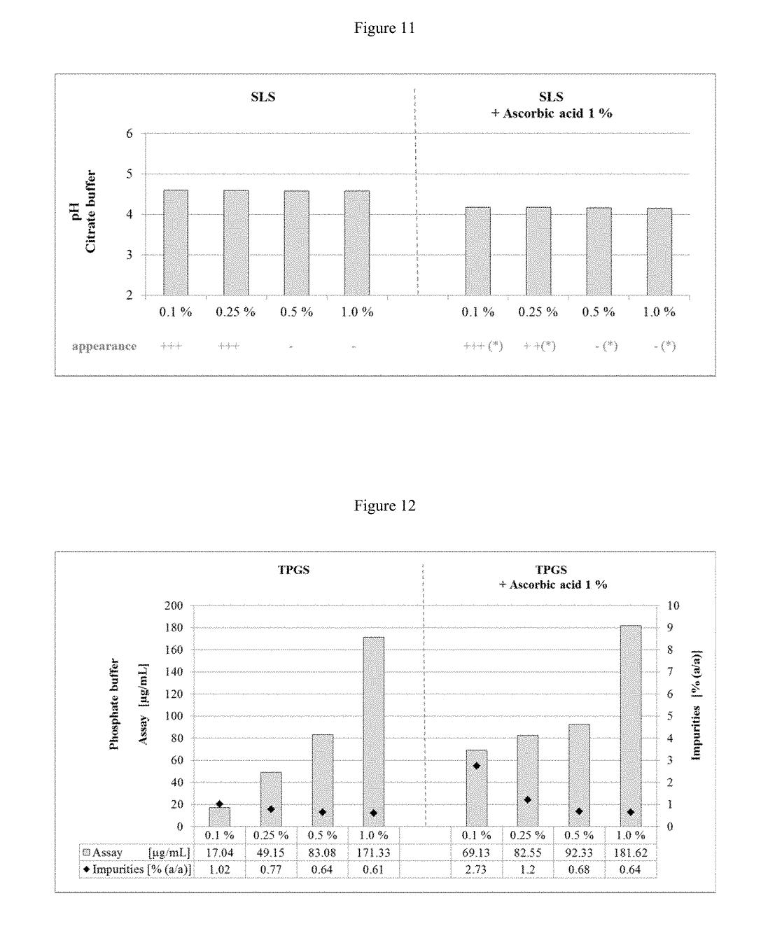

FIG. 10 shows the results of the assay and impurities in citrate buffer and SLS (0.1%; 0.25%; 0.5% and 1.0%) in presence and absence of ascorbic acid. FIG. 11 shows the pH in citrate buffer and SLS (0.1%; 0.25%; 0.5% and 1.0%) in presence and absence of ascorbic acid (+++=very cloudy; ++=cloudy; −=clear; (*)=yellow color).

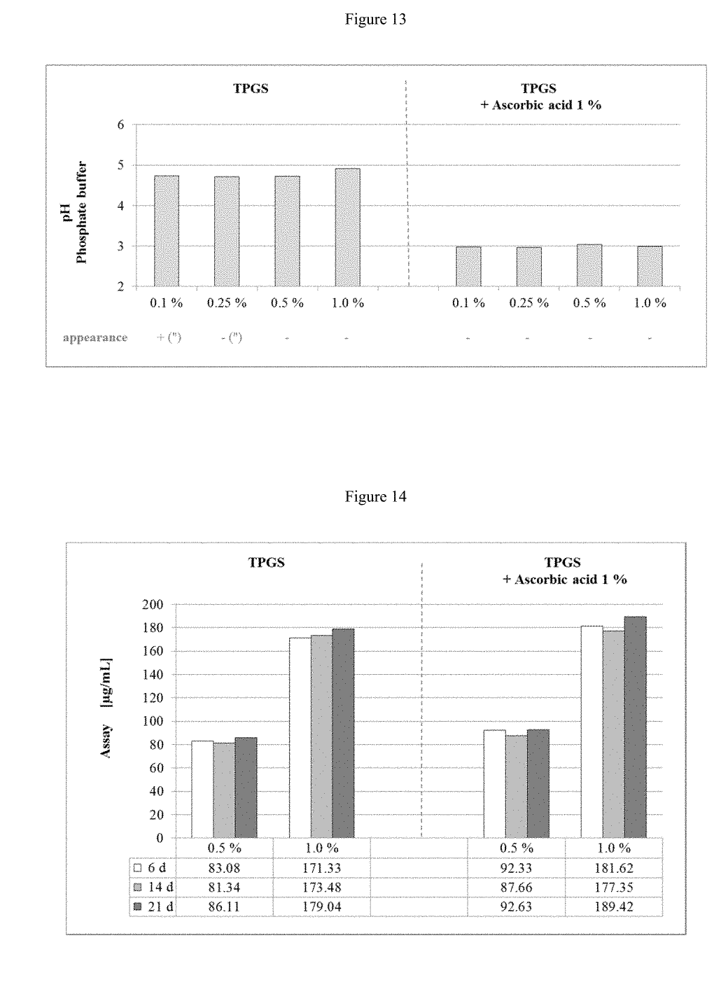

FIG. 12 shows the results of the assay and impurities in phosphate buffer and vitamin E TPGS (0.1%; 0.25%; 0.5% and 1.0%) in presence and absence of ascorbic acid. FIG. 13 shows the pH in phosphate buffer and vitamin E TPGS (0.1%; 0.25%; 0.5% and 1.0%) in presence and absence of ascorbic acid (+=slightly cloudy; −=clear; (″)=visible crystals).

It becomes clear from the data shown in FIGS. 10 to 13 that again, a surfactant concentration-dependent increase in the assay with simultaneous decrease of the impurities of API was observed for both buffer/surfactant combinations (FIG. 10 and FIG. 12). The preparations containing ascorbic acid 1% showed a decrease in their pH values (FIG. 11 and FIG. 13), where the shift was only slightly obtained in citrate buffer/SLS (pH 4.6 to 4.2) and stronger in phosphate buffer/vitamin E TPGS (pH 4.7 to 3.0). The preparations containing ascorbic acid 1% showed no considerable benefit in the impurity profiles of the investigated compositions, whereas the assay of API increased in phosphate buffer in presence of ascorbic acid, which is likely to be caused by the lower pH of the compositions.

Also, the compositions containing citrate buffer and lower concentrations of SLS (0.1% and 0.25%) appeared cloudy (in absence and presence of ascorbic acid), while all combinations of citrate buffer, SLS and ascorbic acid resulted in compositions with yellow color. The observed low assay values with lower surfactant concentrations are likely to be the result of a lower solubility of API, since these compositions appeared cloudy or API crystals were macroscopic visible (FIG. 11 and FIG. 13).

API in phosphate buffer and vitamin E TPGS 0.5% and 1% showed very low impurity levels, even at acidic conditions (FIG. 12).

### EXAMPLE 6—SHORT TERM STABILITY OF API IN PRESENCE AND ABSENCE OF ASCORBIC ACID

The compositions containing phosphate buffer and vitamin E TPGS were investigated in a short term stability study. The aim was to evaluate the possible benefit of an antioxidative effect, provided by the presence of ascorbic acid. The compositions, which had been stirred for six days, were stirred for another 15 days under light protection, resulting in an overall stirring period of 21 days. Samples were analyzed (assay and impurities) after 14 and 21 days (additionally to t=6 days). Visual appearance and pH values were evaluated only after 14 days.

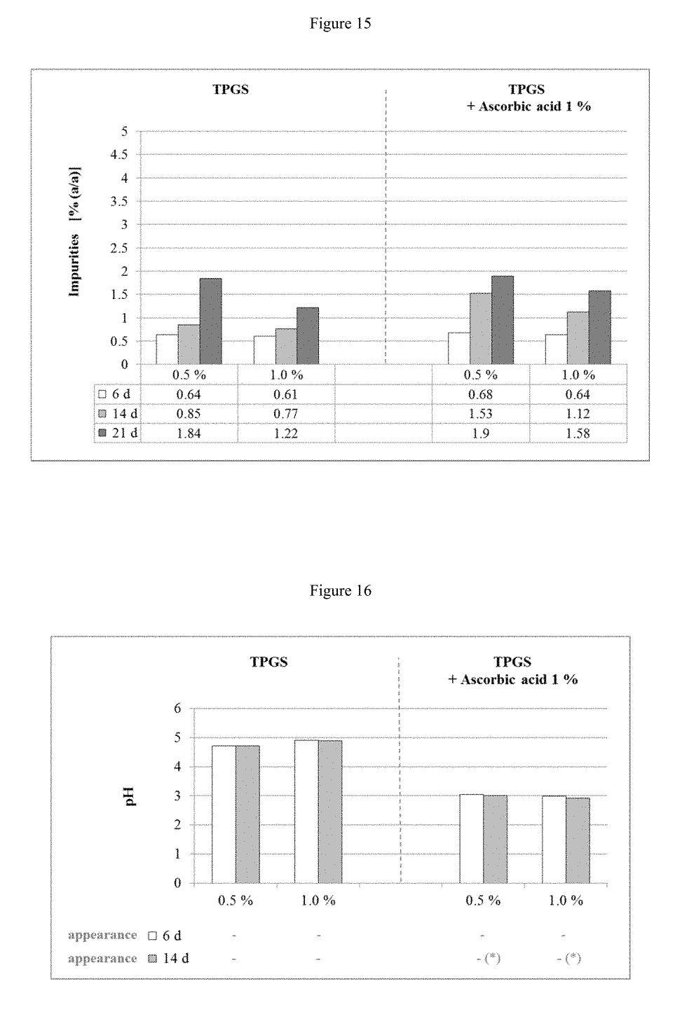

FIG. 14 shows the short term stability of API in phosphate buffer and vitamin E TPGS 0.5% and 1% in presence and absence of ascorbic acid; Assay at t=6, 14 and 21 days. FIG. 15 shows the short term stability of API in phosphate buffer and vitamin E TPGS 0.5% and 1% in presence and absence of ascorbic acid; Impurities at t=6, 14 and 21 days. FIG. 16 shows the short term stability of API in phosphate buffer and vitamin E TPGS 0.5% and 1% in presence and absence of ascorbic acid; pH and appearance at t=6, 14 and 21 days (−=clear; (*)=yellow color).

As shown in FIG. 14 and FIG. 15, for both, assay and impurities of API, no apparent effect of ascorbic acid could be observed over the time period of 21 days. pH values remained constant, as well, while a slight yellow discoloration of ascorbic acid containing preparations occurred (FIG. 16).

### EXAMPLE 7—INFLUENCE OF ASCORBIC ACID ON THE pH

After introduction of ascorbic acid as an antioxidant, a shift in pH and therefore differences in solubility of API were observed (see FIG. 12 and FIG. 13). To investigate the influence of ascorbic acid on the assay and purity of the compositions in dependence of the pH, new batches were manufactured, for which pH values were adjusted to specific values (pH 3, 5 and 7) prior to dissolution of API and overnight stirring. In addition to the immediate measurement of assay and impurities, pH values were measured after one day.

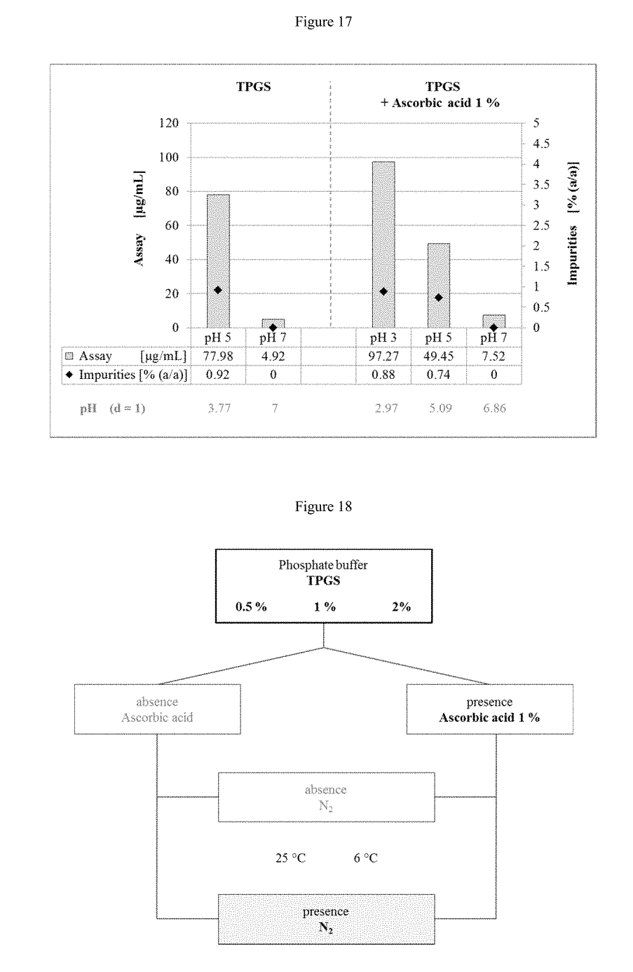

FIG. 17 shows the results of the assay, impurities and pH of API in phosphate buffer and vitamin E TPGS 0.5% in presence and absence of ascorbic acid.

It becomes clear from the data shown in FIG. 17 that the addition of ascorbic acid to the compositions led to a decrease in pH, which necessitated pH adjustment in order to maintain a specific pH value. Since the solubility of API is pH-dependent, assay results but also the impurities were higher for lower pH values (FIG. 17). Higher impurity values were assumed being a result of probable instability of API under acidic conditions. As it offered a good compromise between solubility and stability of API, pH 5 was selected as value for further investigations.

### EXAMPLE 8—EVALUATION OF THE BENEFIT OF NITROGEN-GASSING ON OXIDATIVE STABILITY OF API

A possible beneficial effect of nitrogen-gassing during manufacturing and storage was assessed. Selected compositions were manufactured by dissolution of API via stirring for 24 h and stored after treatment with/without nitrogen at 25° C. or 6° C. for up to 28 days.

FIG. 18 shows a flow chart for assessment of the influence of nitrogen-gassing.

The manufactured batches consisted of phosphate buffer and vitamin E TPGS and were compared to compositions containing ascorbic acid 1%, additionally. API was used at 0.01 wt.-%, 0.02 wt.-% or 0.04 w.-%. The compositions were saturated with API, where 10 mg, 20 mg or 40 mg were dissolved in 100 g buffer containing 0.5 wt.-%, wt.-1% or 2 wt.-% vitamin E TPGS, respectively. The compositions were transferred into polystyrene bottles and stored at 25° C. or 6° C. A part of the compositions was treated with nitrogen during manufacturing and gassed with nitrogen prior to sealing (see FIG. 18). The compositions were tested for assay and purity of API after manufacturing, as well as after 7, 14 and 28 days.

The following table shows the assay results of stored compositions in absence and presence of nitrogen (Asc.=ascorbic acid; −=absence; 1%=presence; N₂=nitrogen; −=absence; +=presence):

| | | | | Assay [μg/mL] | | | | | | |
|---|---|---|---|---|---|---|---|---|---|---|
| vitamin | | | | | 25° C. | | | 6° C. | | |
| E | | | API | | | t = | t = | | t = | t = |
| TPGS | Asc. | N₂ | [%] | t = 0 | t = 7 d | 14 d | 28 d | t = 7 d | 14 d | 28 d |
| 0.5% | 1% | − | 0.01% | 66.81 | 65.66 | 65.27 | 61.46 | 66.21 | 66.54 | 66.15 |
| | | + | | 66.61 | 63.63 | 61.53 | 63.95 | 65.65 | 65.65 | 65.82 |
| | — | − | | 50.22 | 50.56 | 50.23 | 50.36 | 50.43 | 50.71 | 50.27 |
| | | + | | 49.49 | 49.76 | 49.65 | 49.80 | 49.69 | 49.97 | 49.80 |
| 1% | 1% | − | 0.02% | 110.52 | 112.15 | 113.59 | 103.54 | 112.48 | 112.95 | 112.97 |
| | | + | | 110.99 | 107.03 | 106.77 | 93.34 | 109.93 | 110.67 | 110.25 |
| | — | − | | 92.32 | 93.12 | 93.69 | 93.67 | 92.96 | 93.12 | 93.21 |
| | | + | | 93.15 | 93.61 | 93.28 | 93.95 | 93.47 | 93.72 | 93.93 |
| 2% | 1% | − | 0.04% | 218.96 | 222.30 | 213.85 | 197.06 | 222.15 | 226.12 | 225.09 |
| | | + | | 217.91 | 209.88 | 210.66 | 190.68 | 218.73 | 218.92 | 219.23 |
| | — | − | | 212.92 | 214.19 | 213.44 | 215.45 | 214.61 | 214.30 | 214.68 |
| | | + | | 214.85 | 215.81 | 215.34 | 217.45 | 216.81 | 216.54 | 216.93 |

The following table shows the purity results of stored compositions in absence and presence of nitrogen (Asc.=ascorbic acid; −=absence; 1%=presence; N₂=nitrogen; −=absence; +=presence):

| | | | | Impurities [% (a/a)] | | | | | | |
|---|---|---|---|---|---|---|---|---|---|---|
| | | | | | 25° C. | | | 6° C. | | |
| vitamin E | | | API | | | t = | t = | | t = | t = |
| TPGS | Asc. | N₂ | [%] | t = 0 | t = 7 d | 14 d | 28 d | t = 7 d | 14 d | 28 d |
| 0.5% | 1% | − | 0.01% | 0.66 | 0.53 | 1.09 | 1.36 | 0.70 | 0.83 | 0.70 |
| | | + | | 0.58 | 1.00 | 1.74 | 1.82 | 0.65 | 29.21 | 0.70 |
| | — | − | | 0.68 | 0.45 | 1.13 | 1.06 | 0.63 | 0.94 | 0.52 |
| | | + | | 0.71 | 0.36 | 1.19 | 0.87 | 0.73 | 0.83 | 0.54 |
| 1% | 1% | − | 0.02% | 0.55 | 1.01 | 1.05 | 1.63 | 0.67 | 0.96 | 0.91 |
| | | + | | 0.62 | 1.04 | 1.27 | 1.79 | 0.73 | 0.90 | 0.91 |
| | — | − | | 0.63 | 0.74 | 1.01 | 0.80 | 0.66 | 0.82 | 1.15 |
| | | + | | 0.63 | 0.69 | 0.86 | 0.85 | 0.64 | 0.75 | 0.58 |
| 2% | 1% | − | 0.04% | 0.63 | 0.92 | 1.58 | 1.67 | 0.75 | 0.85 | 0.79 |
| | | + | | 0.62 | 1.22 | 1.08 | 1.54 | 0.72 | 0.95 | 0.68 |
| | — | − | | 0.60 | 0.54 | 0.80 | 0.67 | 0.37 | 0.72 | 0.49 |
| | | + | | 0.58 | 0.57 | 0.82 | 0.72 | 0.53 | 0.71 | 0.46 |

The data in the above tables reveal that solubility of API was dependent on the surfactant concentration, hence 0.5 wt.-% vitamin E TPGS resulted in the lowest and 2 wt.-% in the highest assay values. Compositions with 0.5 wt.-% and 1 wt.-% vitamin E TPGS and ascorbic acid showed a slightly higher assay than compositions without ascorbic acid, regardless of the storage temperature. This could be a result of a slightly decreased pH in presence of ascorbic acid, since API solubility is pH-dependent. The treatment with nitrogen resulted in no apparent effect with respect to the assay of API, which remained constant over the investigated time period of 28 days. However, an increasing trend of the impurity profiles was manifested at the storage temperature of 25° C. Especially, ascorbic acid containing compositions with 0.5 wt.-% and 1 wt.-% vitamin E TPGS showed higher degradation of API over time, whereas compositions with vitamin E TPGS 1 wt.-% and 2 wt.-% and without ascorbic acid remained stable.

Impurity formation remained relatively stable at 6° C., where no differences occurred in absence and presence of ascorbic acid. At a storage temperature of 6° C. the sum of all impurities remained below 1% (a/a) for all compositions. Again, treatment with nitrogen provided no evident benefit, regardless of the storage temperature.

### EXAMPLE 9—PRELIMINARY STABILITY STUDY

Two dose strengths were specified: 40 μg/mL and 150 μg/mL API. These concentrations were defined in order to stay below 80% of the saturated solubility values obtained during previous experiments. To enable dissolution of the API, 0.5 wt.-% and 2 wt.-% vitamin E TPGS were used for 40 μg/mL and 150 μg/mL API, respectively (24 h stirring). The compositions were treated with nitrogen and stored at 5° C., 25° C. and 40° C. In parallel, compositions containing 1 wt.-% ascorbic acid were prepared and stored only at 5° C., since previous studies showed adverse effects on the stability of API at 25° C. in combination with ascorbic acid (see FIG. 15).

The following table shows the assay results of preliminary stability study for API intravesical solution (Asc.=ascorbic acid; −=absence; n.d.=not determined):

| vitamin | API | | Assay [% L.S.] | | | | | | | | | |
|---|---|---|---|---|---|---|---|---|---|---|---|---|
| E | [μg/ | | | 6° C. | | | 25° C. | | | 40° C. | | |
| TPGS | mL] | Asc. | t = 0 | 2 w | 1 m | 3 m | 2 w | 1 m | 3 m | 2 w | 1 m | 3 m |
| 0.5% | 40 | — | 54.58 | 54.81 | 54.62 | 55.05 | 54.71 | 54.98 | 54.50 | 54.61 | 54.06 | 52.07 |
| 2% | 150 | | 61.73 | 62.29 | 61.93 | 62.05 | 61.97 | 62.07 | 61.96 | 61.71 | 61.37 | 59.41 |

| | | | | | | | | |
|---|---|---|---|---|---|---|---|---|
| 0.5% | 40 | 1% | 70.39 | 70.56 | 70.25 | n.d. | n.d. | n.d. |
| 2% | 150 | | 68.21 | 68.11 | 67.68 | | | |

The following table shows the purity results of preliminary stability study for API intravesical solution (Asc.=ascorbic acid; −=absence; n.d.=not determined):

| vitamin | API | | Impurities [% (a/a)] | | | | | | | | | |
|---|---|---|---|---|---|---|---|---|---|---|---|---|
| E | [μg/ | | | 6° C. | | | 25° C. | | | 40° C. | | |
| TPGS | mL] | Asc. | t = 0 | 2 w | 1 m | 3 m | 2 w | 1 m | 3 m | 2 w | 1 m | 3 m |
| 0.5% | 40 | — | 0.55 | 0.08 | 0.58 | 0.69 | 0.97 | 0.86 | 1.42 | 1.04 | 1.78 | 4.94 |
| 2% | 150 | | 0.44 | 0.49 | 0.72 | 0.72 | 0.68 | 1.00 | 1.37 | 1.16 | 1.84 | 4.09 |

| | | | | | | | | |
|---|---|---|---|---|---|---|---|---|
| 0.5% | 40 | 1% | 0.57 | 0.57 | 1.00 | n.d. | n.d. | n.d. |
| 2% | 150 | | 0.68 | 0.68 | 1.16 | | | |

The data in the above tables reveal that assay values for all investigated compositions remained stable during the time period of 3 months, when stored at 6° C. or 25° C. However, the assay decreased considerably at 40° C. A trend of increasing degradation of API was observed at 25° C. and was even more pronounced at 40° C. after three months.

The above experimental data demonstrate that excipients provide a concentration-dependent, beneficial effect on the solubility and stability of API, where the combination of phosphate buffer and vitamin E TPGS offered the most promising results. Furthermore, API degradation could be inhibited by light protection during manufacturing and storage of compositions. The implementation of ascorbic acid as antioxidant necessitated pH adjustment due to the occurrence of a pH shift to more acidic pH values and furthermore, resulted in negative effects on the stability at 25° C. The usage of nitrogen as protective gas showed no apparent advantage over the storage time of 3 months. The storage temperature of 6° C. offered a beneficial effect on the stability of the tested compositions.

1. An aqueous pharmaceutical composition comprising cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine or a physiologically acceptable salt thereof at a concentration of at least 5.0 μg/mL.

2. The composition according to claim 1, wherein the concentration is at least 20 μg/ml; preferably at least 40 μg/mL; more preferably at least 80 μg/mL; still more preferably at least 120 μg/mL.

3. The composition according to claim 1, which has a pH value within the range of from pH 2.0 to pH 12; preferably from pH 2.5 to pH 8; more preferably from pH 3.0 to pH 7.0; still more preferably from pH 3.5 to pH 6.5.

4. The composition according to claim 1, which contains a buffer; preferably a buffer derived from phosphoric acid.

5. The composition according to claim 1, which comprises an excipient selected from antioxidants, surfactants and surfactants having antioxidative properties.

6. The composition according to claim 5, wherein the excipient
is non-ionic; and/or
has a HLB value within the range of from 9 to 18; preferably 10 to 17, more preferably 11 to 16, still more preferably 12 to 15.

7. The composition according to claim 5, wherein the excipient comprises a polyethylene glycol residue.

8. The composition according to claim 5, wherein the excipient comprises a vitamin E residue.

9. The composition according to claim 5, wherein the excipient is vitamin E polyethylene glycol succinate.

10. The composition according to claim 5, wherein the concentration of the excipient is within the range of from 0.1 to 5.0 wt.-%; preferably 0.5 to 4.0 wt.-%, more preferably 1.0 to 3.0 wt.-%; in each case relative to the total weight of the composition.

11. The composition according to claim 1, which comprises cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine in form of its free base.

12. The composition according to claim 1, which has a storage stability of at least 6 months.

13. The composition according to claim 1, for use in the treatment of bladder pain syndrome.

14. The composition for use according to claim 13, wherein the composition is administered topically; preferably intravesically.

15. Cis-(E)-4-(3-fluorophenyl)-2′,3′,4′,9′-tetrahydro-N,N-dimethyl-2′-(1-oxo-3-phenyl-2-propenyl)-spiro[cyclohexane-1,1′[1H]-pyrido[3,4-b]indol]-4-amine or a physiologically acceptable salt thereof for use in the treatment of bladder pain syndrome.
